(19) United States
(12) Patent Application Publication
(10) Pub. No.: US 2024/0024149 A1
(43) Pub. Date: Jan. 25, 2024

(54) ERGONOMIC CUSHION BASE

(71) Applicants: Melanie Clair Fahringer, Las Vegas, NV (US); Stephen Anthony Fahringer, Las Vegas, NV (US)

(72) Inventors: Melanie Clair Fahringer, Las Vegas, NV (US); Stephen Anthony Fahringer, Las Vegas, NV (US)

(21) Appl. No.: 18/357,699

(22) Filed: Jul. 24, 2023

Related U.S. Application Data
(60) Provisional application No. 63/391,541, filed on Jul. 22, 2022.

(51) Int. Cl. A61F 5/30

(52) U.S. Cl. CPC A61F 5/30

(57) ABSTRACT

An orthopedic cushion includes an inelastic base having a first flat surface and an upper second surface and a resilient pad having a first surface that ergonomically supports a user's anatomy and a lower second surface connected to the upper surface of the inelastic base. The inelastic base serves as a stable platform that better aligns the hips, legs, and spine and improves circulation while sitting, thereby decreasing back, leg and hip pain.

### CROSS-REFERENCE TO RELATED APPLICATION

This application claims the benefit of priority of U.S. provisional application No. 63/391,541, filed Jul. 22, 2022, the contents of which are herein incorporated by reference.

### BACKGROUND OF THE INVENTION

The present invention relates to ergonomic cushions and, more particularly, to an ergonomic cushion base.

There are a number of “support” type cushions that are designed to improve spinal alignment and take pressure off the hips, legs and back. These devices are soft and cannot give the needed support. Think about putting a soft wrap on a broken bone vs putting on a hard cast. The hard cast will keep the bone aligned properly. The soft wrap will help but does not offer the same amount of support. Regular cushions and comfort seat cushions do not provide the stable platform needed to better support the hips, back and neck.

As can be seen, there is a need for a support cushion with a stable platform.

### SUMMARY OF THE INVENTION

In one aspect of the present invention, an orthopedic cushion comprises an inelastic base having a first planar surface and an upper second surface and a resilient pad having a first surface ergonomically configured to support a user's anatomy and a lower second surface coupled to the upper second surface of the inelastic base.

In another aspect of the present invention, a polymeric base operative to stably support an orthopedic cushion comprises an inelastic polymeric sheet having a first planar surface configured to encompass the lower surface area of the orthopedic cushion.

The hard base greatly improves any cushion's effectiveness by providing the needed foundation to keep and improve posture while sitting, effectively providing an orthotic device. The stable platform better aligns the hips, legs, and spine and improves circulation while sitting, thereby decreasing back, leg and hip pain.

These and other features, aspects and advantages of the present invention will become better understood with reference to the following drawings, description, and claims.

### BRIEF DESCRIPTION OF THE DRAWINGS

FIG. 1 is a perspective view of a cushion base according to an embodiment of the present invention;

FIG. 2 is a sectional view thereof, taken along line 2-2 from FIG. 1;

FIG. 3 is an exploded view thereof; and

FIG. 4 is an enlarged sectional view thereof.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a solid, stable platform for use with, or installation in, a cushion operative to improve posture and correct spinal alignment.

The hard base may be milled or stamped out of a polymer sheet to the specifications of a selected Spinal/orthopedic alignment cushion. The polymer may have any suitable rigidity or durometer reading such that the polymer forms a sturdy, stable base for a cushion upon which a user sits. The base may be permanently affixed to or placed under any comfort, orthopedic, or spinal alignment seat cushion to improve the support provided by the cushion.

The cushion base of the present invention is distinct from the structure of a chair seat and may be placed on any suitable surface.

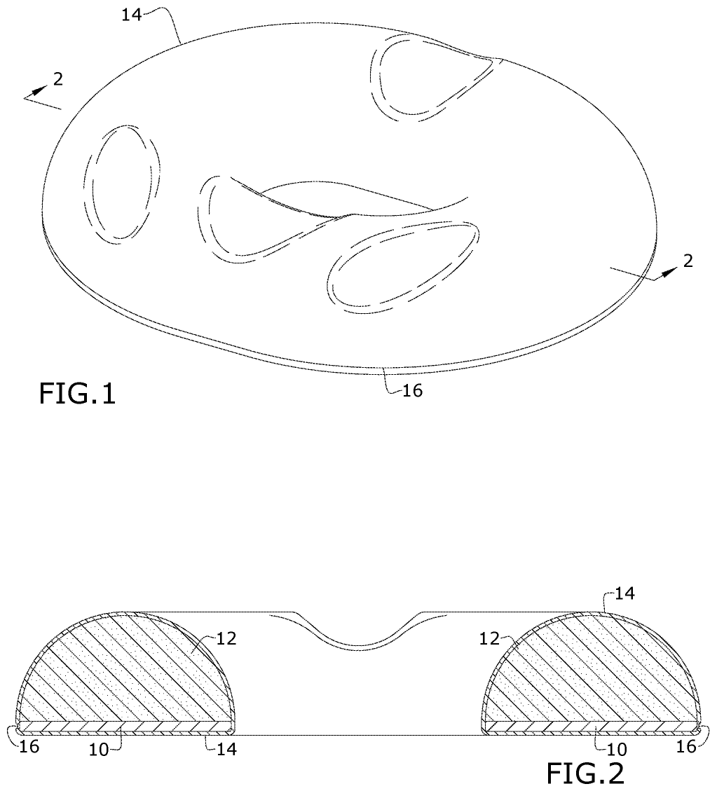

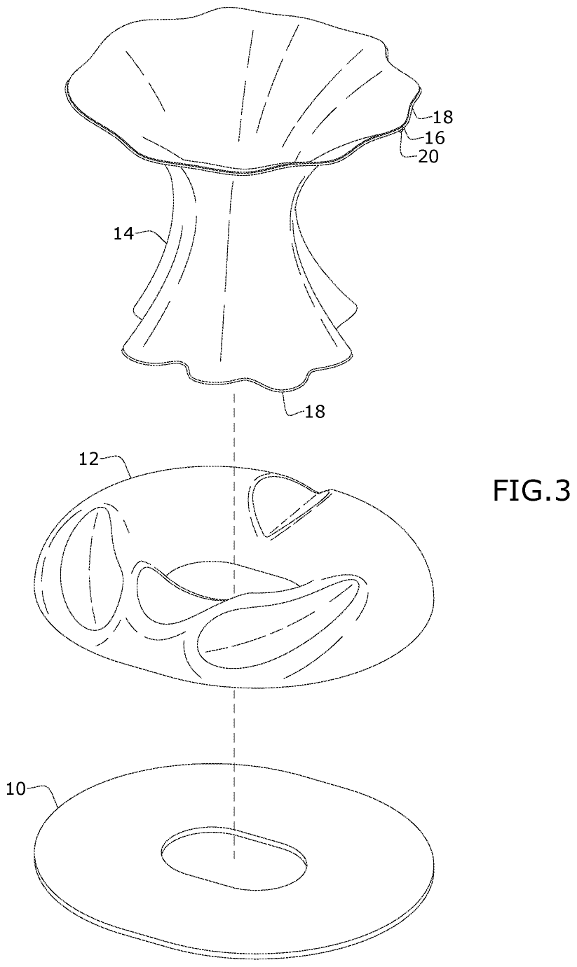

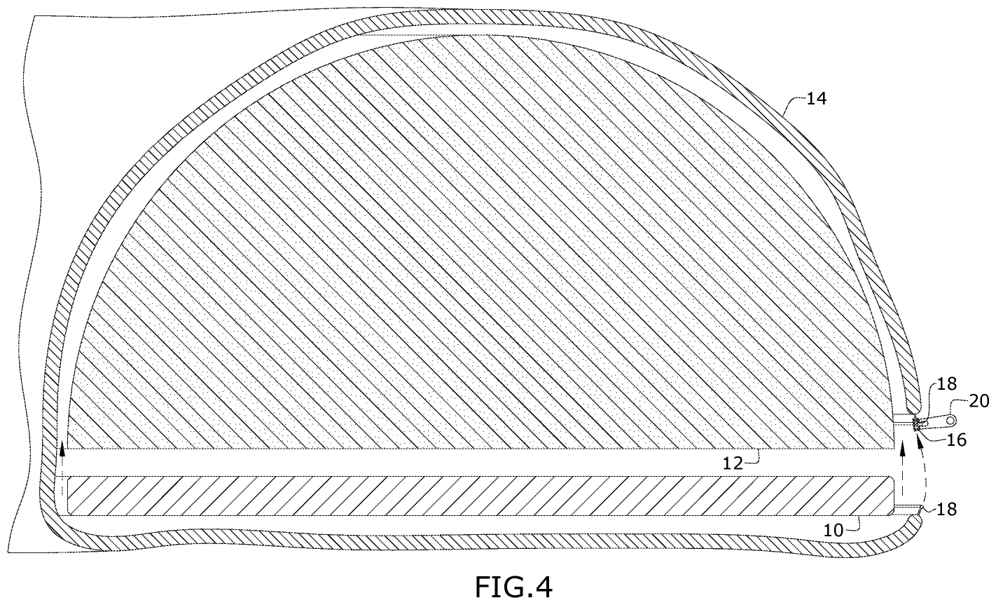

Referring to FIGS. 1 through 4, a seat cushion 12 on a hard base 10 according to an embodiment of the present invention, is shown, encompassed by a cover 14 with a zipper 16. The zipper 16 has zipper teeth 18 and a zipper pull 20. In this embodiment, the assembled base 10, cushion 12, and cover have a donut shape configured to accommodate the user's buttocks and thighs. The donut shape may have any suitably shaped circumference and any suitable inner shape, ranging from circular to rectangular, such as oval or square or any combination thereof. Note that the base of the present invention is not limited to use with or in donut shaped cushions. The cushion 12 is formed with recessed regions on the upper surface (opposite the base) that function to support the user's bottom in a position that better aligns the spine and may relieve pressure on the user's tailbone. The base 10 as shown is planar, with a central aperture. However, the base 10 is not limited to this configuration. The base 10 may have an ergonomic shape to better align the spine and may be covered by a cushion 12 that is not in itself ergonomically configured. The cover 14 has a substantially tubular shape when unzipped and removed from the cushion 12, as shown in FIG. 3.

It should be understood, of course, that the foregoing relates to exemplary embodiments of the invention and that modifications may be made without departing from the spirit and scope of the invention as set forth in the following claims.

What is claimed is:

1. An orthopedic cushion comprising:
a) an inelastic base having a first planar surface and an upper second surface; and
b) a resilient pad having a first surface ergonomically configured to support a user's anatomy and a lower second surface coupled to the upper second surface of the inelastic base.

2. The orthopedic cushion of claim 1, wherein a central aperture is formed in the cushion and base such that the orthopedic cushion has a donut shape.

3. The orthopedic cushion of claim 1, wherein the first surface of the resilient pad has recessed regions configured to accommodate the user's anatomy and/or to relieve pressure on a portion of the user's anatomy.

4. The orthopedic cushion of claim 1, further comprising a cover surrounding the cushion and base.

5. The orthopedic cushion of claim 4, wherein the cover has a double funnel shape, having a relatively narrow tubular central portion, widening to a first end with a substantially circular edge and a second end with a substantially circular edge, wherein the first end and the second end have equal diameters.

6. The orthopedic cushion of claim 5, wherein the cover has mating zipper portions stitched along the substantially circular edges thereof.

7. A polymeric base operative to stably support an orthopedic cushion, comprising:
a) an inelastic polymeric sheet having a first planar surface configured to encompass a lower surface area of the orthopedic cushion.

8. The polymeric base of claim 7, further comprising a second, planar surface.

9. The polymeric base of claim 7, wherein the polymeric base has an oval circumference.

10. The polymeric base of claim 7, having a central aperture formed therein.

11. The polymeric base of claim 7, wherein the polymeric base is affixed to the orthopedic cushion.
